(19) United States
(12) Patent Application Publication
(10) Pub. No.: US 2010/0032617 A1
(43) Pub. Date: Feb. 11, 2010

(54) Process for manufacturing epichlorohydrin

(75) Inventors: Patrick Gilbeau, Braine-le-Comte (BE); Philippe Krafft, Rhode Saint Genese (BE)

Correspondence Address: Solvay;c/o B. Ortego - IAM-NAFTA, 3333 Richmond Avenue, Houston, TX, 77098-3099, US

(73) Assignee: SOLVAY (SOCIETE ANONYME), Brussels (BE)

(21) Appl. No.: 12/527,538

(22) PCT Filed: Feb. 15, 2004
(86) PCT No.: PCT/EP08/51830

§ 371 Date: Aug. 17, 2009

(30) Foreign Application Priority Data
Feb. 20, 2007 (FR) 0753375
Jun. 4, 2007 (FR) 0755448
Sep. 28, 2007 (FR) 0757941

(51) Int. Cl. C09K 3/00; C07D 301/02

(52) U.S. Cl. 252/182.12; 549/518

(57) ABSTRACT

Process for manufacturing epichlorohydrin comprising the following steps:
a) in a liquid reaction medium, a mixture of 1,3-dichloro-2-propanol and 2,3-dichloro-1-propanol, in which the 1,3-dichloro-2-propanol content is at least 10 wt %, is reacted with at least one basic compound in order to form epichlorohydrin and a salt; and
b) at least one part of the liquid reaction medium from step a) is subjected to a settling operation in which at least a first fraction containing most of the epichlorohydrin which was contained in the part of the reaction medium from step a) before the settling operation and a second fraction containing most of the salt which was contained in the part of the reaction medium from step a) before the settling operation are separated.

The present patent application claims the benefit of the following patent applications FR 0753375 filed on 20 Feb. 2007, FR 0755448 filed on 4 Jun. 2007, FR 0757941 filed on 28 Sep. 2007, and of the provisional U.S. patent application No. 61/013704 filed on 14 Dec. 2007, the content of all of which is incorporated here by reference.

The present invention relates to a process for manufacturing epichlorohydrin. The present invention relates more specifically to a process for manufacturing epichlorohydrin via reaction between dichloropropanol and a basic agent.

Epichlorohydrin is a reaction intermediate in the manufacture of epoxy resins, synthetic elastomeres, glycidyl ethers, polyamide resins, etc. (Ullmann's Encyclopedia of Industrial Chemistry, Fifth Edition, Vol. A9, p. 539).

In the process for manufacturing epichlorohydrin from dichloropropanol and a basic agent, the dehydrochlorination of dichloropropanol is accompanied by saponification of some of the epichlorohydrin formed, mainly leading to the formation of glycerol and thus reducing the yield of epichlorohydrin. To overcome this disadvantage, it has been proposed to remove the epichlorohydrin as soon as it is formed by stripping the reaction medium, for example with steam. This way of proceeding generates, however, large volumes of aqueous effluents contaminated by organic substances that it is necessary to treat before disposing thereof (Milchert E. and Goc W., Pol. J. Appl. Chem. 41, 113-118 (1997); Kleiboehmer W., Klumpe M. and Popp W., Gewaesserschutz, Wasser, Abwasser, 200(Wissenschaftlich-Technische Mitteilungen des Instituts zur Foerderung der Wasserguete- und Wassermengenwirtschaft e.V., 2005, v5), 8/1-8/5). In Patent U.S. Pat. No. 3,061,615 in the name of Solvay & Co., it has been proposed to remove the epichlorohydrin as soon as it is formed by extracting the reaction medium with a solvent which dissolves epichlorohydrin and which is immiscible with (insoluble in) water. This way of proceeding has the disadvantage of complicating the process via the introduction of a third substance that it is necessary to separate and recycle.

The objective of the present invention is to provide a process for manufacturing epichlorohydrin from dichloropropanol which does not have these disdvantages, while preserving a high epichlorohydrin selectivity.

The invention hence relates to a process for manufacturing epichlorohydrin comprising the following steps:
a) in a liquid reaction medium, a mixture of 1,3-dichloro-2-propanol and 2,3-dichloro-1-propanol, in which the 1,3-dichloro-2-propanol content is at least 10 wt %, is reacted with at least one basic compound in order to form epichlorohydrin and a salt; and
b) at least one part of the liquid reaction medium from step a) is subjected to a settling operation in which at least a first fraction containing most of the epichlorohydrin which was contained in the part of the reaction medium from step a) before the settling operation and a second fraction containing most of the salt which was contained in the part of the reaction medium from step a) before the settling operation are separated.

In the rest of the document the expression “dichloropropanol” will be used to denote the mixture of 1,3-dichloro-2-propanol and 2,3-dichloro-1-propanol with the exclusion of any other compound.

The expressions “most of the epichlorohydrin” and “most of the salt” are understood to mean half or more than half of the epichlorohydrin or salt which was contained in the part of the reaction medium from step a) before the settling operation.

It has been discovered that when, in the dichloropropanol used, the content of 1,3-dichloro-2-propanol is at least 10 wt %, it is possible to carry out the dehydrochlorination reaction under less severe temperature and residence time conditions so that it is no longer necessary to remove the epichlorohydrin as soon as it is formed. These conditions greatly reduce the secondary reactions which are the origin of the contamination of the aqueous effluents of the process. Without wishing to be tied to any one theoretical explanation, it is believed that these mild reaction conditions are made possible by the high reactivity of the 1,3-dichloro-2-propanol isomer in the reaction of dehydrochlorination by the basic compound. Among the advantages of the process according to the invention, with respect to the classical processes using stripping or solvent extraction which remove the epichlorohydrin as soon as it is formed, mention may be made of:
(A) a lower steam consumption, and therefore an energy saving;
(B) a reduction in the equipment size;
(C) a decrease in the volume of the aqueous effluents to be treated;
(D) the generation of epichlorohydrin-based compositions which may be used without further treatment, like for instance a pre-treatment, in other manufacturing processes; and
(E) the generation of aqueous solutions rich in salts and poor in total organic carbon which may be used for instance as such in electrolysis processes.

In the process according to the invention, the part of the reaction medium from step a) may be subjected to a treatment prior to the settling operation. This treatment may be chosen from the operations of heating, of cooling, of dilution, of addition of a salt, of addition of an acid compound, and combinations of at least two of them.

The addition of an acid compound makes it possible to neutralize the basic compound optionally present in the part of the reaction medium from step a). The amount of acid compound added is generally such that the pH measured in the part of the reaction medium from step a) before the settling operation is between 5 and 9. Such a pH measurement requires that the reaction medium in question be well stirred. It has been found that the basic compound still optionally present in the part of the reaction medium from step a) before the settling operation is capable of promoting epichlorohydrin hydrolysis reactions, resulting in a loss of selectivity.

The acid compound may be chosen from organic and inorganic acids and mixtures thereof. Inorganic acids are preferred. The expression “inorganic acid” is understood to mean acids of which the molecule does not contain a carbon-hydrogen bond, such as hydrogen chloride, sulphuric acid, phosphoric acid and boric acid. Gaseous hydrogen chloride or an aqueous solution of hydrogen chloride are preferred, an aqueous solution of hydrogen chloride being more preferred.

In the process according to the invention, the dichloropropanol from step a) may be derived from several processes such as, for example, the allyl chloride hypochlorination process, the allyl alcohol chlorination process, the glycerol hydrochlorination process, the 2,3-dichloropropionaldehyde hydrogenation process as described in documents WO 1997/48667, U.S. Pat. No. 6,350,922 and U.S. Pat. No. 5,744,655, the 1,2-dichlorethylene hydroformylation process as described in document WO 2005/116004, the 1,3-dichloroacetone hydrogenation process as described in documents WO 2005/097722 and WO 2003/064357. 2,3-dichloropropionaldehyde may itself be obtained by chlorination of acrolein and/or hydroformylation of 1,2-dichloroethylene as described in documents U.S. Pat. No. 2,860,146 and WO 2005/116004. 1,3-dichloroacetone may itself be obtained by chlorination of acetone and/or by bromine/chlorine exchange starting from 1,3-dibromoacetone as described in Applications WO 2005/097722 and WO 2005/115954. Acrolein may be obtained by selective oxidation of propylene. 1,2-dichloroethylene may be a by-product of the synthesis of vinyl chloride starting from ethane and/or be obtained by chlorination of acetylene. Acetylene may be obtained by conventional processes such as hydrolysis of calcium carbide and/or pyrolysis of hydrocarbons, crude oil and even coal, such as described in “Industrial Organic Chemistry, Third, Completely Revised Edition, VCH, 1997, pp. 93-98”. 1,3-dibromoacetone may be obtained by bromination of acetone, as described in document WO 2005/115954. Acetone may itself be obtained by conventional processes, such as, for example, oxidation of propylene, dehydrogenation of isopropanol and/or decomposition of cumene hydroperoxide, as described in “Industrial Organic Chemistry, Third, Completely Revised Edition, VCH, 1997, pp. 276-277 and 347-355”.

In the process according to the invention, at least one part of the dichloropropanol is preferably obtained by reaction between glycerol and a chlorinating agent and/or by reaction between allyl chloride and a hypochlorinating agent and/or by reaction between allyl alcohol and a chlorinating agent and/or by reaction between 2,3-dichloropropionaldehyde and a hydrogenating agent and/or by reaction between 1,2-dichloroethylene and a hydroformylating agent and/or by reaction between 1,3-dichloroacetone and a hydrogenating agent.

In the process according to the invention, the dichloropropanol is preferably obtained by reaction between glycerol and a chlorinating agent and/or by reaction between allyl chloride and more preferably by reaction between glycerol and a chlorinating agent as described in Patent Applications WO 2005/054167, WO 2006/100311, WO 2006/100312, WO 2006/100313, WO 2006/100314, WO 2006/100315, WO 2006/100316, WO 2006/100317, WO 2006/106153, WO 2007/054505, WO 2006/100318, WO 2006/100319, WO 2006/100320, WO 2006/106154, WO 2006/106155 and FR 06/05325, all filed in the name of Solvay S A.

In the process according to the invention, when at least one part of the dichloropropanol is obtained by reaction between glycerol and a chlorinating agent, the chlorinating agent preferably contains hydrogen chloride such as described in Patent Application WO 2005/054167 by Solvay S A. The hydrogen chloride may be in the form of gas or an aqueous solution of hydrogen chloride or a mixture of the two, preferably in the form of gas or a mixture of gas and an aqueous solution of hydrogen chloride. Glycerol may be obtained from fossil or renewable raw materials. It is preferred to use glycerol obtained from renewable materials. A glycerol which is particularly suitable may be obtained during the conversions of fats or oils of vegetable or animal origin, such as saponification, transesterification or hydrolysis reactions. A particularly suitable glycerol may be obtained during the conversion of animal fats. Another particularly suitable glycerol may be obtained during the manufacture of biodiesel. Another particularly suitable glycerol may be obtained during the fatty acid manufacture.

In the process according to the invention, the dichloropropanol may be dichoropropanol extrinsic to the process according to the invention, recycled dichloropropanol or a mixture of the two. The expression “recycled dichloropropanol” is understood to mean dichloropropanol which has been separated in a step subsequent to step b) in the process according to the invention and which has then been recycled to step a) of said process. The term “extrinsic dichloropropanol” is understood to mean dichloropropanol which has not been recycled in the process according to the invention.

In the process according to the invention, the extrinsic dichloropropanol content in the dichloropropanol is generally at least 40 wt %, preferably at least 80 wt %, more preferably at least 90 wt % and most particularly preferably at least 95 wt %. A dichloropropanol essentially composed of extrinsic dichloropropranol is very suitable.

In the process according to the invention, the dichloropropanol generally contains at least 300 g of 1,3-dichloro-2-propanol/kg of dichloropropanol, more specifically at least 400 g/kg, especially at least 750 g/kg, in many cases at least 800 g/kg, particularly at least 900 g/kg, and preferably at least 920 g/kg. This content of 1,3-dichloro-2-propanol in the dichloropropanol is generally at most 990 g/kg and usually at most 960 g/kg. Contents of 925, 930, 935, 940, 945, 950 or 955 g/kg are particularly convenient. It is also possible to use a dichloropropanol composed essentially of 1,3-dichloro-2-propanol.

In the process according to the invention, the extrinsic dichloropropanol has a ratio of the 1,3-dichloro-2-propanol content to the 2,3-dichloro-1-propanol content which is generally at least 0.11, preferably at least 0.43, more preferably at least 0.66 and most particularly preferably at least 4. This ratio is generally at most 99.

In the process according to the invention, the ratio of the 2,3-dichloro-1-propanol content to the 1,3-dichloro-2-propanol content in the recycled dichloropropanol is generally higher than this ratio observed in the extrinsic dichloropropanol. It is at least equal to the latter. In one particular embodiment, this ratio is greater than or equal to 0.06, for instance greater than or equal to 0.1 and in special cases greater than or equal to 0.5. That ratio is usually less than or equal to 10, particularly less than or equal to 8 preferably less than or equal to 5 andin most preferred cases less than or equal to 2. Ratios of 0.6, 0.7, 0.8, 0.9, 1.0, 1.1, 1.2, 1.3, 1.4, 1.5, 1.6, 1.7, 1.8 and 1.9 are particularly convenient. In another embodiment, this ratio is greater than 10, preferably greater than or equal to 15. That ratio is in general less than or equal to 120, advantageously less than or equal to 100.

In the process according to the invention, the reaction medium may contain water. The water may be introduced with the dichloropropanol. In this case, the water content introduced by the dichloropropanol relative to the sum of the water content introduced by the dichloropropanol and the dichloropropanol content is generally at least 5 g of water/kg, preferably at least 20 g/kg and most particularly preferably at least 50 g/kg. This water content is generally at most 850 g/kg.

In the process according to the invention, the liquid reaction medium may also contain carboxylic acids. These acids may be introduced with the dichloropropanol and be such as those mentioned as catalysts for the reaction between glycerol and a chlorinating agent in Application WO 2005/054167 in the name of Solvay S A or such as those mentioned as catalysts for the reaction between a polyhydroxylated aliphatic hydrocarbon and hydrogen chloride in Application WO 2006/020234 or such as those mentioned as catalysts for the reaction between glycerol and hydrogen chloride in Application WO 2006/020234. In this case in point, the content of carboxylic acids relative to the sum of the content of carboxylic acids introduced by the dichloropropanol and the dichloropropanol content, is generally less than 10 mol %, usually less than 3 mol %, preferably less than 0.1 mol % and most particularly preferably less than 0.001 mol %.

In the process according to the invention, the liquid reaction medium may also contain mineral acids such as, for example, hydrogen chloride. These acids may be introduced by the dichloropropanol. The hydrogen chloride content relative to the sum of the hydrogen chloride content introduced by the dichloropropanol and the dichloropropanol content is generally at most 50 wt %, ususally at most 25 wt %, preferably at most 2 wt % and most particularly preferably at most 0.01 wt %.

In the process according to the invention, the liquid reaction medium may also contain other organic compounds than dichloropropanol, epichlorohydrin and organic acids. These organic compounds may, for example, be derived from dichloropropanol synthesis processes such as, for example, glycerol, monochloropropanediol, glycerol esters, monochloropropanediol esters, dichloropropanol esters, partially chlorinated and/or esterified glycerol oligomers, aldhehydes, acrolein, chloroacetones and especially 1-chloroacetone. The content of these compounds relative to the sum of the content of organic compounds introduced by the dichloropropanol and the dichloropropanol content is generally at most 100 g/kg, preferably at most 50 g/kg and most particularly preferably at most 20 g/kg.

In the process according to the invention, the basic compound from step a) may be an organic or inorganic basic compound. Organic basic compounds are for example amines, phosphines and ammonium, phosphonium or arsonium hydroxides. Inorganic basic compounds are preferred. The expression “inorganic compounds” is understood to mean compounds which do not contain a carbon-hydrogen bond. The inorganic basic compound may be chosen from alkali and alkaline-earth metal oxides, hydroxides, carbonates, hydrogencarbonates, phosphates, hydrogenphosphates and borates, and mixtures thereof. Alkali and alkaline-earth metal oxides and hydroxides are preferred.

In the process according to the invention, the basic compound may be in the form of a liquid, an essentially anhydrous solid, a hydrated solid, an aqueous and/or organic solution or an aqueous and/or organic suspension. The basic compound is preferably in the form of an essentially anhydrous solid, a hydrated solid, an aqueous solution or an aqueous suspension.

The expression “essentially anhydrous solid” is understood to mean a solid of which the water content is less than or equal to 20 g/kg, preferably less than or equal to 10 g/kg and more preferably less than or equal to 1 g/kg.

The expression “hydrated solid” is understood to mean a solid of which the water content is at least 20 g/kg and at most 700 g/kg, preferably at least 50 g/kg and at most 650 g/kg and most particularly preferably at least 130 g/kg and at most 630 g/kg. The hydrates which denote solid combinations of substances with one or more water molecules are examples of hydrated solids.

When the basic compound is used in the form of an aqueous solution, its content in the aqueous solution is generally greater than 20 g/kg, preferably greater than or equal to 70 g/kg and more preferably greater than or equal to 150 g/kg. This content is generally less than or equal to the solubility of the basic solid in water at the reaction temperature of step a).

When the basic compound is used in the form of an aqueous suspension, its content in the aqueous suspension is generally greater than the solubility of the basic solid in water at the reaction temperature of step a), preferably greater than or equal to 20 g/kg and more preferably greater than or equal to 70 g/kg. This content is generally less than or equal to 400 g/kg, preferably less than 300 g/kg.

The preferred basic compounds are in the form of concentrated aqueous solutions or suspensions of sodium hydroxide or calcium hydroxide or in the form of purified caustic brine.

The sodium hydroxide content of solutions or suspensions of sodium hydroxide is generally greater than or equal to 30 g/kg, usually greater than or equal to 40 g/kg, particularly greater than or equal to 60 g/kg, in many cases greater than or equal to 100 g/kg, and preferably greater than or equal to 120 g/kg. This sodium hydroxide content is generally less than or equal to 300 g/kg, commonly less than or equal to 250 g/kg, often less than or equal to 200 g/k and advantageously less than or equal to 160 g/kg. Contents of 125, 130, 135, 140, 145, 150 and 155 g/kg are particularly convenient.

The expression “purified caustic brine” here means sodium hydroxide which contains sodium chloride such as, for example, that produced in a diaphragm electrolysis process. The sodium hydroxide content of the purified caustic brine is generally greater than or equal to 30 g/kg, preferably greater than or equal to 40 g/kg and more preferably greater than or equal to 60 g/kg. This sodium hydroxide content is generally less than or equal to 300 g/kg, preferably less than or equal to 250 g/kg and more preferably less than or equal to 200 g/kg. The sodium chloride content of the purified caustic brine is generally greater than or equal to 30 g/kg, preferably greater than or equal to 50 g/kg and more preferably greater than or equal to 70 g/kg. This sodium chloride content is generally less than or equal to 250 g/kg, preferably less than or equal to 200 g/kg and more preferably less than or equal to 180 g/kg.

It is also possible to use a mixture of several basic agents as a function of the availabilities and of the economic optimization of the industrial site where the process according to the invention is established. The basic agents preferred for producing these mixtures are limewater and solutions of sodium hydroxide and of purified caustic brine, for example, a mixture of limewater and a sodium hydroxide solution, a mixture of limewater and purified caustic brine. These mixtures may be produced in any relative proportion of at least two of these basic agents. They may be produced before introduction into the liquid reaction medium and also in this medium.

In the process according to the invention, the water content of the liquid reaction medium at step a) is generally less than or equal to 950 g/kg of liquid reaction medium, preferably less than or equal to 800 g/kg and particularly preferably less than or equal to 700 g/kg. This water content is generally greater than or equal to 100 g/kg of liquid reaction medium, preferably greater than 200 g/kg and most particularly preferably greater than 350 g/kg.

In a first embodiment of the process according to the invention, in step a), the dichloropropanol is used in stoichiometric or substoichiometric amounts with respect to the effective amount of the basic compound. The expression “effective amount of the basic compound” is understood to mean the amount of basic compound reduced to the amount required for the reaction with the organic and mineral acids optionally present in the reaction medium. In this case, at least 1 effective equivalent of basic compound per equivalent of dichloropropanol is generally used. At least 1.2 effective equivalents of basic coumpound per equivalent of dichloropropanol are usually used and at least 1.5 effective equivalents of basic compound per equivalent of dichloropropanol are frequently used and at most 5 effective equivalents of basic compound per equivalent of dichloropropanol are generally used.

In a second embodiment of the process according to the invention, which is preferred, in step a), the dichloropropanol is used in excess with respect to the effective amount of the basic compound. In this case, at most 0.99 effective equivalent of basic compound per equivalent of dichloropropanol is generally used. At most 0.95 effective equivalent of basic compound per equivalent of dichloropropanol is usually used, at most 0.8 effective equivalent of basic compound is frequently used and at the minimum 0.2 effective equivalent of basic compound is used. The advantage of working with a deficit of basic compound with respect to the dichloropropanol makes it possible to reduce the epichlorohydrin degradation reactions (especially the hydrolysis reactions) during steps (a) and (b). The settling operation may hence be carried out over longer time periods, favourable to better separation of the first and second fractions.

The liquid reaction medium from step a) may contain an organic solvent. All organic substances that dissolve epichlorohydrin and that are not, or not very, miscible with water may be used as solvent. The expression “organic substances that are not, or not very, miscible with water” is understood to mean organic substances whose solubility in water at 25° C. is at most 50 g/kg. These compounds do not comprise the reactants used and the products formed during the reaction from step a) of the process. The solvent content of the liquid reaction medium from step a) expressed as the weight ratio between the solvent and the dichloropropanol is generally less than or equal 9, commonly less than or equal to 8, often less than or equal to 5, particularly less than or equal to 2, in many cases less than or equal to 1, very often less than or equal to 0.8, advantageously less than or equal to 0.5, for instance less than or equal to 0.3 and preferably less than or equal to 0.1. The solvent content of the liquid reaction medium from step a) is commonly less than or equal to 80 wt % of dichloropropanol, usually less than or equal to 50 wt %, in many cases less than or equal to 30 wt % and preferably less than or equal to 10 wt %. The solvent content of the liquid reaction medium from step a) is generally higher than or equal to 0.01 wt % of dichloropropanol, frequently higher than or equal to 0.1 wt %, often higher than or equal to 1 wt % and advantageously higher than or equal to 5 wt %. Most particularly preferably, the liquid reaction medium from step a) does not contain an organic solvent, i.e. has a solvent content less than 0.01 wt % of dichloropropanol. The content of dichloropropanol to be referred to is the content before the reaction of step a).

Step a) may be carried out in batch, semi-continuous or continuous mode. The continuous mode, in which the reaction medium from step a) is continuously supplied and drawn off, is preferred.

In the process according to the invention, the reaction from step a) is generally carried out at a temperature of at most 100° C., usually of at most 90° C., frequently of at most 80° C., often of at most 65° C. andvery often of at most 50° C. This reaction temperature is generally at least 0° C., frequently at least 10° C., often at least 15° C., in many cases of at least 30° C., and advantageously of at least 40° C. Temperatures of 41, 42, 43, 44, 45, 46, 47, 48 and 49° C. are particularly convenient.

In the process according to the invention, the reaction from step a) is generally carried out at a pressure of at most 20 bar absolute, preferably of at most 15 bar absolute and particularly preferably of at most 10 bar absolute. This reaction pressure is generally at least 0.01 bar absolute, preferably at least 0.1 bar absolute and more particularly preferably at least 0.2 bar absolute. A pressure between 0.6 and 1.4 bar absolute is particularly suitable. A pressure between 0.7 and 1.3 bar absolute is particularly convenient. Pressures of 0.8, 0.9, 1.0, 1.1 and 1.2 bar absolute are more particularly convenient.

The reactor may be a plug-flow type, stirred tank type or recycle loop type reactor. It may be in the form of a plate column with stirring on each plate. The reactants may be introduced separately or premixed.

The reaction may be carried out adiabatically by regulating the reactor operating temperature via control of the temperature of the reactants. The reaction may also be carried out isothermally with regulation of the reactor operating temperature via control of the temperature of the reactants and by means of heat exchange. Heat exchange may be achieved using a jacket, an internal heat exchanger or an external heat exchanger.

The reaction from step a) may be carried out with vigorous stirring so as to ensure good mutual dispersion of the dichloropropanol and the basic agent, or in the absence of stirring. All stirring methods are suitable: stirring in the reactor by means of blades, turbines or by means of exterior shuttles using a pump.

A favourable selectivity for the formation of epichlorohydrin is obtained in a stirred reactor in batch mode or in a continuous stirred reactor.

When step a) of the process according to the invention is carried out in batch mode or in a plug-flow reactor, the reaction time is generally at least 1 min, usually at least 2 min and frequently at least 5 min. This time is generally at most 240 min, usually at most 180 min, frequently at most 150 min and more specifically at most 130 min.

When step a) of the process according to the invention is carried out in continuous mode, the residence time, defined as the ratio of the volume of the reaction liquid to the total volume flow rate of the liquid reactants, is generally at least 1 min, usually at least 4 min and frequently at least 7 min. This residence time is generally at most 240 min, usually at most 180 min, frequently at most 150 min, more specifically at most 60 min, in many cases at most 30 min, advantageously at most 20 min and specifically at most 10 min.

The temperature, time, stirring and composition of the medium are generally adjusted to obtain a conversion of the reactant in deficit, the dichloropropanol or the basic compound, of at least 20%, often of at least 30%, frequently of at least 40%, in many cases of at least 50%, advantageously of at least 75% and specifically of at least 90%.

In the process according to the invention, the settling operation from step b) may be carried out by gravity or by centrifugation. Settling by gravity is preferred.

In the process according to the invention, the settling operation from step b) is generally carried out at a temperature of at least 0° C., frequently of at least 5° C. often of at least 20° C., very often of at least 30° C. and advantageously of at least 50° C. This reaction temperature is generally at most 100° C., often at most 85° C., in many cases at most 75° C. and advantageously of at most 60° C.

In the process according to the invention, the settling operation from step b) is generally carried out at a pressure of at most 20 bar absolute, preferably of at most 15 bar absolute and particularly preferably of at most 10 bar absolute. This reaction pressure is generally at least 0.01 bar absolute, preferably at least 0.1 bar absolute and more particularly preferably at least 0.2 bar absolute. A pressure between 0.6 and 1.4 bar absolute is particularly suitable. A pressure between 0.7 and 1.3 bar absolute is particularly convenient. Pressures of 0.8, 0.9, 1.0, 1.1 and 1.2 bar absolute are more particularly convenient.

Step b) may be carried out in batch, semi-continuous or continuous mode. Continuous mode is preferred.

When the settling operation from step b) is carried out in batch mode, the settling operation is carried out over a time generally of at least 5 min and usually of at least 10 min. The duration of the settling operation from step b) is generally at most 120 min.

When the settling operation from step b) is carried out in continuous mode, the settling operation may be carried out with residence times that are identical or optionally different for each of the phases in the settling tank. These residence times are generally at least 5 min, usually at least 10 min. The duration of the settling operation from step b) is generally at most 120 min.

In the process according to the invention, the difference in density between the first and second fractions separated in step b) is at least 0.001, often at least 0.002, in many cases at least 0.01 and in particular of at least 0.05. That difference in density is usually lower than or equal to 0.2. Differences of 0.06, 0.07, 0.08, 0.09, 0.1, 0.11, 0.12, 0.13, 0.14, 0.15, 0.16, 0.17, 0.18 and 0.19 are particularly suitable.

The difference in density between the two fractions is governed independently by the nature and the content of the organic constituents from the first fraction and by the salinity of the second fraction. The density of the first fraction may be increased by reducing the degree of epichlorohydrin formation in step a) or by reintroducing some 1,3-dichloro-2-propanol and/or some 2,3-dichloro-1-propanol between step a) and step b). Preferably, the densest phase is the first fraction. When the salt in the second fraction is sodium chloride, a 20 wt % salt content in the second fraction enables separation of the two fractions in all cases. With a 25 wt % salt content in the second fraction, it is necessary that the total concentration of 1,3-dichloro-2-propanol and 2,3-dichloro-1-propanol in the first fraction be greater than at least 15% so that the first fraction has the highest density.

The first fraction separated in step b) generally contains at least 100 g of epichlorohydrin/kg of first fraction, preferably at least 200 g/kg, even more preferably at least 300 g/kg, still more preferably at least 400 g/kg, more particularly preferably at least 500 g/kg, even more particularly preferably at least 600 g/kg, still more particularly preferably at least 700 g/kg, most particularly preferably at least 800 g/kg and very most particularly preferably at least 850 g/kg. The epichlorohydrin content of the first fraction separated is generally at most 900 g/kg. The epichlorohydrin content of the first fraction separated depends, for example, on the use of an organic solvent and/or on an incomplete conversion of the mixture of 1,3-dichloro-2-propanol and 2,3-dichloro-1-propanol.

The first fraction separated in step b) generally contains at most 2 g of chloroacetone/kg of first fraction and preferably at most 0.3 g/kg, more preferably at most 0.1 g/kg, and most particularly preferably at most 0.05 g/kg. The chloroacetone content is generally at least 0.005 g/kg.

The first fraction separated in step b) generally contains at most 5 g of acrolein/kg of first fraction and preferably at most 0.3 g/kg and more preferably at most 0.1 g/kg. The acrolein content is generally at least 0.07 g/kg.

The first fraction separated in step b) generally contains at most 20 g of chloroethers/kg of first fraction, preferably at most 5 g/kg, more preferably at most 2 g/kg, and most particularly preferably at most 1 g/kg. The content of chloroethers is generally at least 0.5 g/kg.

Chloroethers are compounds of which the molecule comprises at least one chlorine atom and at least one oxygen atom, this oxygen atom being bonded to two carbon atoms. Epichlorohydrin is not considered here as a chloroether. These chloroethers preferably contain six carbon atoms. These chloroethers preferably contain two, sometimes three, chlorine atoms. These chloroethers preferably contain two oxygen atoms. These chloroethers are preferably chosen from compounds of crude chemical formula: C₆H₁₀Cl₂O₂, C₆H₁₂Cl₂O, C₆H₉Cl₃O₂, C₆H₁₁Cl₃O₂ and mixtures of at least two of them.

The first fraction separated in step b) generally contains at most 10 g of chloroether of crude formula C₆H₁₀Cl₂O₂/kg of first fraction, preferably at most 5 g/kg, more preferably at most 0.5 g/kg, and most particularly preferably at most 0.1 g/kg. The content of this chloroether is generally at least 0.05 g/kg.

The first fraction separated in step b) generally contains at most 5 g of chloroether of crude formula C₆H₁₂Cl₂O/kg of first fraction, preferably at most 2 g/kg, more preferably at most 0.5 g/kg, and most particularly preferably at most 0.1 g/kg. The content of this chloroether is generally at least 0.05 g/kg.

The first fraction separated in step b) generally contains at most 5 g of chloroether of crude formula C₆H₉Cl₃O₂/kg of first fraction, preferably at most 2 g/kg, more preferably at most 0.5 g/kg, and most particularly preferably at most 0.1 g/kg. The content of this chloroether is generally at least 0.02 g/kg.

The first fraction separated in step b) generally contains at most 5 g of chloroether of crude formula C₆H₁₁Cl₃O₂/kg of first fraction, preferably at most 2 g/kg, even more preferably at most 1 g/kg, and most particularly preferably at most 0.6 g/kg. The content of this chloroether is generally at least 0.5 g/kg.

The first fraction separated in step b) generally contains other organic compounds such as, for example, 1,3-dichloro-2-propanol, 2,3-dichloro-1-propanol and mixtures thereof. The sum of the contents of these dichloropropanols is generally less than or equal to 900 g/kg of first fraction, preferably less than or equal to 800 g/kg, more preferably less than or equal to 700 g/kg, even more preferably less than or equal to 500 g/kg, still more preferably less than or equal to 300 g/kg, particularly preferably less than or equal to 200 g/kg and particularly preferably less than or equal to 150 g/kg. The sum of the contents of these dichloropropanols is generally at least 90 g/kg. Values of that sum of 100, 110, 120, 130 and 140 g/kg are particularly convenient The ratio between the 2,3-dichloro-1-propanol and the 1,3-dichloro-3-propanol is usually greater than or equal to 0.06, often greater than or equal to 0.1 and frequently greater than or equal to 0.5. That ratio is usually less than or equal to 10, generally less than or equal to 8, in many cases less than or equal to 5 and in particular less than or equal to 2. Ratios of 0.6, 0.7, 0.8, 0.9, 1.0, 1.1, 1.2, 1.3, 1.4, 1.5, 1.6, 1.7, 1.8 and 1.9 are particularly convenient.

The first fraction separated in step b) generally contains other organic compounds in addition to the epichlorohydrin, chloroacetone, acrolein, chloroethers and dichloropropanols.

The latter may come from the dichloropropanol manufacturing process and/or be formed during the reaction between dichloropropanol and the basic compound during step a) of the process according to the invention. Examples of these compounds are glycerol, 3-chloro-1,2-propanediol, 2-chloro-1,3-propanediol, and mixtures thereof, hydroxyacetone, glycidol, methyl glycidyl ether, 1,2,3-trichloropropane, cis and trans 1,3-dichloropropenes, 1,3-dichloropropane and 2-chloro-2-propen-1-ol.

The sum of the contents of glycerol, hydroxyacetone and glycidol is generally at most 100 g/kg of first fraction, frequently at most 50 g/kg, usually at most 30 g/kg, in particular at most 10 g/kg and more specifically at most 1 g/kg. The sum of these contents is generally at least 0.1 g/kg.

The sum of the contents of 3-chloro-1,2-propanediol and 2-chloro-1,3-propanediol is generally at most 5 g/kg of first fraction, preferably at most 3 g/kg, and more preferably at most 1 g/kg. This sum is generally at least 0.5 g/kg.

The methyl glycidyl ether content is generally at most 5 g/kg of first fraction, preferably at most 3 g/kg, and more preferably at most 1 g/kg. This content is generally at least 0.005 g/kg.

The 1,2,3-trichloropropane content is generally at most 10 g/kg of first fraction, preferably at most 5 g/kg, more preferably at most 3 g/kg and most particularly preferably at most 1 g/kg. This content is generally at least 0.01 g/kg.

The sum of the contents of cis and trans 1,3-dichloropropenes is generally at most 2 g/kg of first fraction, preferably at most 1 g/kg, and more preferably at most 0.1 g/kg. This sum is generally at least 0.01 g/kg.

The 1,3-dichloropropane content is generally at most 2 g/kg of first fraction, preferably at most 1 g/kg, and more preferably at most 0.5 g/kg. This content is generally at least 0.01 g/kg.

The 2-chloro-2-propen-1-ol content is generally at most 2 g/kg of first fraction, preferably at most 1 g/kg, and more preferably at most 0.5 g/kg. This content is generally at least 0.01 g/kg.

The first fraction separated in step b) generally contains water and inorganic compounds such as the basic compound and the salt. The water content is generally at most 90 g/kg of first fraction, frequently at most 80 g/kg, usually at most 50 g/kg, more specifically at most 30 g/kg and even more specifically at most 15 g/kg. The water content is generally at least 1 g/kg of first fraction. The salt content is generally at most 10 g/kg of first fraction, frequently at most 5 g/kg, usually at most 2 g/kg, more specifically at most 0.1 g/kg and even more specifically at most 0.015 g/kg. This salt content is generally at least 0.01 g/kg.

The first fraction separated in step b) may be used as a reactant in a process for manufacturing epoxy derivatives such as epoxy resins, glycidyl ethers, such as cresyl glycidyl, butyl, decyl or dodecyl ethers, glycidyl esters such as glycidyl acrylates and methacrylates, synthetic glycerol, polyamide-epichlorohydrin resins, products which will be used in food and drink applications such as chemical formulations for water treatment, for instance polyacrylamides, polyamines and quaternary ammonium salts, resins for the production of water-resistant paper, epichlorohydrin elastomers, such as epichlorohydrin homopolymers, epichlorohydrin/ethylene oxide copolymers and epichlorohydrin/ethylene oxide/allyl glycidyl ether terpolymers, surfactants, flame retardants, such as phosphorylated flame retardants, cationization agents or detergent ingredients.

The invention also relates to an organic composition of which the epichlorohydrin content is at least 100 g/kg and at most 900 g/kg of composition and of which the chloroacetone content is at least 0.005 g/kg and at most 2 g/kg of composition, possibly being obtained according to the process described above, in which the first fraction separated in step b) constitutes the organic composition.

The invention also relates to the use of this organic composition in processes for manufacturing epoxy derivatives such as epoxy resins, glycidyl ethers, such as cresyl glycidyl, butyl, decyl or dodecyl ethers, glycidyl esters such as glycidyl acrylates and methacrylates, synthetic glycerol, polyamide-epichlorohydrin resins, products which will be used in food and drink applications such as chemical formulations for water treatment, for instance polyacrylamides, polyamines and quaternary ammonium salts, resins for the production of water-resistant paper, epichlorohydrin elastomers, such as epichlorohydrin homopolymers, epichlorohydrin/ethylene oxide copolymers and epichlorohydrin/ethylene oxide/allyl glycidyl ether terpolymers, surfactants, flame retardants, such as phosphorylated flame retardants, cationization agents or detergent ingredients.

The invention also relates to an organic composition of which the epichlorohydrin content is at least 100 g/kg and at most 900 g/kg of composition and of which the chloroacetone content is at least 0.005 g/kg and at most 2 g/kg of composition.

In the process according to the invention, the salt included in the second fraction separated in step b) may be an organic or inorganic salt. Inorganic salts are preferred. The expression “inorganic salts” is understood to mean salts whose constituent ions do not contain a carbon-hydrogen bond.

In the process according to the invention, the second fraction separated in step b) generally comprises water. The water content is generally at least 500 g of water per kg of second fraction, preferably at least 600 g/kg, more preferably at least 700 g/kg and more particularly preferably at least 750 g/kg. The water content is generally at most 990 g of water per kg of second fraction, preferably at most 950 g/kg, more preferably at most 900 g/kg and more particularly preferably at most 850 g/kg.

In the process according to the invention, the second fraction separated in step b) generally comprises at least 50 g of salt/kg, preferably at least 100 g of salt/kg, more preferably at least 150 g of salt/kg and most particularly preferably at least 200 g of salt/kg. Most particularly, the salt concentration is below the solubility limit of the salt in this second fraction. This is because salt precipitation complicates the process. This precipitation may lead to blockages of the installation and to trapping of organic compounds in the precipitated salt crystals. It has been found that it is possible to remain below the solubility limit of the salt in the second fraction separated in step b), by addition of water, depending on the overall balance of water introduced with the reactants, in step a) and/or between step a) and b) and/or in step b). Introduction with the reactants by dilution of these in step a) is an easy way to avoid salt precipitation in the second fraction separated in step b).

The advantage of a salt content at the limit of its solubility in the second fraction separated in step b) is two-fold. It makes it possible, on the one hand, to reduce the concentration of organic compounds in the second fraction (salting-out effect) and, on the other hand, to reduce the water content of the first fraction.

The salts present in the second fraction separated in step b) of the process according to the invention are preferably chosen from alkali and alkaline-earth metal chlorides, sulphates, hydrogensulphates, hydroxides, carbonates, hydrogencarbonates, phosphates, hydrogenphosphates and borates, and mixtures thereof. A portion of these salts cannot be produced in the course of the reaction between dichloropropanol and the basic agent during step a) of the process according to the invention. These salts may thus be present in the reactants, for example. The term “reactants” is understood to mean dichloropropanol and the basic compound. The salts may also be added to step a) or to step b) of the process according to the invention, before the settling operation. Preferably, these salts are partly formed in the reaction of step a) and are partly present in the basic compound.

In the process according to the invention, the second fraction may contain organic compounds. The latter may come from the dichloropropanol manufacturing process and/or be formed during the reaction between dichloropropanol and the basic compound during step a) of the process according to the invention. Examples of these compounds are epichlorohydrin, 1,3-dichloro-2-propanol, 2,3-dichloro-1-propanol, glycerol, 3-chloro-1,2-propanediol, 2-chloro-1,3-propanediol, chloroacetone, hydroxyacetone, glycidol and 2-chloro-2-propen-1-ol.

The epichlorohydrin content of the second fraction separated in step b) is generally at least 0.1 g/kg of second fraction, preferably at least 1 g/kg, more preferably at least 5 g/kg and most particularly preferably at least 10 g/kg. This content does not generally exceed 60 g/kg, preferably 50 g/kg, even more preferably 40 g/kg and most particularly preferably 35 g/kg.

The sum of the 1,3-dichloro-2-propanol and 2,3-dichloro-1-propanol contents of the second fraction separated in step b) is generally at least 0.1 g/kg of second fraction, preferably at least 1 g/kg and more preferably at least 2 g/kg. This sum is generally at most 100 g/kg, preferably at most 80 g/kg and even more preferably at most 40 g/kg.

The sum of the 3-chloro-1,2-propanediol and 2-chloro-1,3-propanediol contents of the second fraction separated in step b) is generally at most 50 g/kg of second fraction, preferably at most 10 g/kg and even more preferably at most 1 g/kg. This sum is generally at least 0.1 g/kg.

In the process according to the invention, the second fraction separated may contain a basic compound, preferably an inorganic basic compound. This inorganic basic compound may be chosen from alkali or alkaline-earth metal oxides, hydroxides, carbonates, hydrogencarbonates, phosphates, hydrogenphosphates and borates, and mixtures of at least two of them. The inorganic basic compound content is generally at least 0.1 g/kg of second fraction, preferably at least 0.5 g/kg, and more preferably at least 1 g/kg. This content is generally at most 25 g/kg of second fraction, preferably at most 10 g/kg, and more preferably at most 5 g/kg.

The total organic carbon (TOC) content of the second fraction separated in step b) is generally at most 40 g of carbon/kg of second fraction separated in step b) and frequently at most 16 g/kg and usually at most 13 g/kg.

The density of the second fraction separated in step b) is generally at least 1.03, preferably at least 1.07 and more particularly preferably at least 1.11. The density is generally at most 1.28, preferably at most 1.21, even more preferably at most 1.20 and most particularly preferably at most 1.19.

The second fraction separated in step b) may be conveyed for instance as such to an electrolysis process. The electrolysis process is, for example, a process for producing chlorine and sodium hydroxide, when the inorganic salt is sodium chloride, for example.

The sodium hydroxide produced in such a process may advantageously be recycled to step a) of the process according to the invention.

The chlorine produced in such a process may advantageously be used in a synthesis for hydrochloric acid production or of which the hydrochloric acid is one of the co-products. This hydrochloric acid may be used as a raw material in the process for synthesis of dichloropropanol.

The invention also relates to an aqueous composition of which the salt content is greater than or equal to 50 g/kg of composition and of which the epichlorohydrin content is at least 0.1 g/kg and at most 60 g/kg, possibly being obtained according to the process described above, in which the second fraction separated in step b) constitutes the aqueous composition. The aqueous composition may comprise, besides a salt and epichlorohydrin, 1,3-dichloro-2-propanol and 3-chloro-1,2-propanediol. The salt content is at least 50 g/kg, preferably at least 100 g/kg, particularly preferably at least 150 g/kg and most particularly preferably at least 200 g/kg. The epichlorohydrin content is at least 0.1 g/kg, preferably at least 1 g/kg and particularly preferably at least 2 g/kg. The epichlorohydrin content is at most 60 g/kg, preferably at most 50 g/kg, particularly preferably at most 40 g/kg and most particularly preferably at most 35 g/kg. The 1,3-dichloro-2-propanol content is at least 0.1 g/kg, preferably at least 1 g/kg and particularly preferably at least 2 g/kg. The 1,3-dichloro-2-propanol content is at most 100 g/kg, preferably at most 80 g/kg and particularly preferably at most 40 g/kg. The 3-chloro-1,2-propanediol content is at most 50 g/kg, preferably at most 10 g/kg and particularly preferably at most 1 g/kg. The 3-chloro-1,2-propanediol content is at least 0.1 g/kg. The density of the aqueous composition is at least 1.03, preferably at least 1.07 and particularly preferably at least 1.11. The density is at most 1.28, preferably at most 1.21, more preferably at most 1.20 and particularly preferably at most 1.19.

The invention also relates to the use of this aqueous composition in an electrolysis process.

The invention also relates to an aqueous composition of which the salt content is greater than or equal to 50 g/kg and of which the epichlorohydrin content is at least 0.1 g/kg and at most 60 g/kg.

In step b) of the process according to the invention, it is also possible to separate a third fraction. This third fraction is generally composed of one or more of the salts as defined above.

The process according to the invention may comprise at least one supplementary step between step a) and step b).

This supplementary step may be a filtration or centrifugation step. A filtration step is preferred. This filtration step makes it possible to remove solid compounds which could hamper the settling step b). These solids are, for example, the salts formed during the reaction from step a) or introduced with the reactants such as defined above. The latter case is encountered more particularly when the basic compound is limewater, which may contain salts that are not very soluble, such as calcium carbonate or calcium sulphate.

This supplementary step may also consist of the addition of an organic solvent such as defined above. It is preferred not to add organic solvent between the reaction step a) and the settling step b) of the process according to the invention.

The examples below are intended to illustrate the invention without, however, limiting it.

### EXAMPLE 1

### According to the Invention

A 1-litre glass thermostated reactor was charged with 258.76 g of 1,3-dichloro-2-propanol (2.01 mol). Added to the flask over 20 min, at 25° C. and with vigorous stirring, were 397.1 g of a 19.1 wt % aqueous solution of NaOH (1.90 mol). At the end of the addition, the resulting mixture was transferred into a separating funnel. 179.39 g of a first fraction, of which the density was 1.185, and 488.95 g of a second fraction, of which the density was 1.182, were recovered. The compositions, expressed in g/kg of the first and second fractions separated, are given in Table 1 (M.C.=main constituent).

The proportion of epichlorohydrin in the second fraction separated only represents 3.3% of the total epichlorohydrin formed. The overall epichlorohydrin selectivity is 94.0% with respect to the base consumed.

TABLE 1

| | First fraction | Second fraction |
|---|---|---|
| Constituent | separated | separated |
| Water | 13 | M.C. |
| NaCl | 0.041 | 226.5 |
| NaOH | | 0.16 |
| Epichlorohydrin | 891 | 11.1 |
| 1,3-dichloro-2-propanol | 95 | 2.5 |
| 3-chloro-1,2-propanediol | 0.2 | 0.44 |
| Glycerol | <0.1 | |
| Chloroacetone | <0.1 | — |
| Hydroxyacetone | <0.1 | <0.01 |
| Glycidol | <0.1 | 2.6 |
| TOC (g C/l) | | 8.7 |

### EXAMPLE 2

### According to the Invention

A 1-litre glass thermostated reactor was charged with 258 g of 1,3-dichloro-2-propanol (2.0 mol) and 73.2 g of water. Added to the flask over 20 min, at 5° C. and with vigorous stirring, were 213 g of a 30 wt % aqueous solution of NaOH (1.60 mol). After a supplementary stirring period of 35 min, the resulting mixture was transferred into a separating funnel. 206.4 g of a first fraction, of which the density was 1.23, and 324.7 g of a second fraction, of which the density was 1.18, were recovered. The compositions, expressed in g/kg of the first and second fractions separated, are given in Table 2 (M.C.=main constituent).

The proportion of epichlorohydrin in the second fraction separated only represents 1.3% of the total epichlorohydrin formed. The overall epichlorohydrin selectivity is 99.5% with respect to the base consumed.

TABLE 2

| | First fraction | Second fraction |
|---|---|---|
| Constituent | separated | separated |
| Water | 21 | M.C. |
| NaCl | 0.036 | 215.2 |
| NaOH | | 0.24 |
| Epichlorohydrin | 703 | 6.6 |
| 1,3-dichloro-2-propanol | 275 | 11 |
| 3-chloro-1,2-propanediol | 0.3 | 3.2 |
| Glycerol | — | 0.2 |
| Chloroacetone | <0.01 | — |
| Hydroxyacetone | 0.02 | <0.01 |
| Glycidol | 0.5 | 3 |
| TOC (g C/l) | | 9.6 |

### EXAMPLE 3

### According to the Invention

A 1-litre glass thermostated reactor was charged with 258 g of 1,3-dichloro-2-propanol (2.0 mol) and 123.1 g of water. Added to the flask over 20 min, at 45° C. and with vigorous stirring, were 213 g of a 30 wt % aqueous solution of NaOH (1.60 mol). After a supplementary stirring period of 2 min, the resulting mixture was transferred into a separating funnel. 194.2 g of a first fraction, of which the density was 1.23, and 393.9 g of a second fraction, of which the density was 1.19, were recovered. The compositions, expressed in g/kg of the first and second fractions separated, are given in Table 3.

The proportion of epichlorohydrin in the second fraction separated only represents 1.9% of the total epichlorohydrin formed. The epichlorohydrin selectivity is 94.7% with respect to the base consumed.

TABLE 3

| | First fraction | Second fraction |
|---|---|---|
| Constituent | separated | separated |
| Water | 21 | M.C. |
| NaCl | 0.015 | 226.5 |
| NaOH | | 0.16 |
| Epichlorohydrin | 708 | 6.7 |
| 1,3-dichloro-2-propanol | 271 | 9 |
| 3-chloro-1,2-propanediol | 0.3 | 2.2 |
| Glycerol | — | 0.1 |
| Chloroacetone | 0.05 | — |
| Hydroxyacetone | 0.02 | <0.01 |
| Glycidol | 0.06 | 2.6 |

### EXAMPLE 4

### According to the Invention

A 1-litre glass thermostated reactor was charged with 56.2 g of 88 wt % CaO (1.77 mol) and 200.2 g of water. Added to the flask over 1 min, at 45° C. and with vigorous stirring, were 258 g of 1,3-dichloro-2-propanol (2.0 mol) preheated to the reaction temperature. After a supplementary stirring period of 120 min, the resulting mixture was filtered over porous glass and the filtrate was transferred into a separating funnel. 17.5 g of wet solid, 177.7 g of a first fraction, of which the density was 1.206, and 292.2 g of a second fraction, of which the density was 1.278, were recovered. The compositions, expressed in g/kg of the first and second fractions separated, are given in Table 4.

The solid dried at 100° C. weighs 9.6 g; it contains 45 wt % of calcium, 29 wt % of chloride and its basicity, expressed as CaO, is 22.1%. The proportion of epichlorohydrin in the second fraction separated only represents 2.2% of the total epichlorohydrin formed. The epichlorohydrin selectivity is 90.8% with respect to the mineral chloride formed.

TABLE 4

| | First fraction | Second fraction |
|---|---|---|
| Constituent | separated | separated |
| Water | 12 | M.C. |
| CaCl₂ | 0.025 | 297 |
| CaO | | 1.2 |
| Epichlorohydrin | 759 | 10.4 |
| 1,3-dichloro-2-propanol | 228 | 1.9 |
| 3-chloro-1,2-propanediol | 0.7 | 0.14 |
| Glycerol | 0.4 | — |
| Chloroacetone | 0.05 | — |
| Hydroxyacetone | 0.02 | 0.03 |
| Glycidol | 1.4 | 0.01 |
| TOC (g C/l) | | 12 |

### EXAMPLES 5 TO 9

### According to the Invention

A 72-ml glass thermostated jacketed reactor was continuously supplied with sodium hydroxide and with dichloropropanol. The reaction medium was constantly stirred vigorously. The liquid mixture exiting the reactor by continuous overflow was collected and then separated in batch mode in a glass funnel so as to obtain a first fraction and a second fraction. The reaction temperature, residence time, sodium hydroxide content, dichloropropanol composition, flow rates of the reactants, compositions and densities of the organic and aqueous phases, pH of the aqueous phase, and the degrees of conversion of the sodium hydroxide and of the 2 dichloropropanol isomers are given in Table 6.

TABLE 6

| Trial No. | | 5 | 6 | 7 | 8 | 9 |
|---|---|---|---|---|---|---|
| Reactor volume | (ml) | 72 | 72 | 72 | 72 | 72 |
| Temperature | (° C.) | 25 | 25 | 25 | 25 | 65 |
| Residence time | (min) | 14.4 | 20 | 20 | 20 | 20 |
| Implementation | | | | | | |
| NaOH content in | (g/kg) | 188.2 | 185.4 | 185.4 | 185 | 185 |
| the aqueous sodium | | | | | | |
| hydroxide | | | | | | |
| Flow rate of | (g/h) | 226.2 | 162.4 | 155.2 | 157.1 | 157.1 |
| aqueous sodium | | | | | | |
| hydroxide | | | | | | |
| 1,3-dichloro-2- | (g/kg) | 1000 | 1000 | 1000 | 500 | 500 |
| propanol content in | | | | | | |
| dichloropropanol | | | | | | |
| 2,3-dichloro-1- | (g/kg) | | | | 500 | 500 |
| propanol content in | | | | | | |
| dichloropropanol | | | | | | |
| Flow rate of | (g/h) | 151.3 | 109.0 | 117.2 | 117.2 | 117.2 |
| dichloropropanol | | | | | | |
| Base/(1,3- | (mol/mol) | 0.91 | 0.89 | 0.79 | 0.8 | 0.8 |
| dichloro-2-propanol + | | | | | | |
| 2,3-dichloro-1- | | | | | | |
| propanol) | | | | | | |
| Production | | | | | | |
| pH measured on | | 13.1 | 13.0 | 12.6 | 13.8 | 11.5 |
| exiting the reactor | | | | | | |
| First fraction | | | | | | |
| separated | | | | | | |
| Flow rate | (g/h) | 102.8 | 71.6 | 83.1 | 94 | 80.4 |
| Density | (g/ml) | 1.2 | 1.184 | 1.204 | 1.209 | 1.223 |
| Epichlorohydrin | (g/kg) | 864 | 862 | 741 | 501 | 667 |
| 1,3-dichloro-2- | (g/kg) | 116 | 118 | 233 | 4 | 6 |
| propanol (1,3-D) | | | | | | |
| 2,3-dichloro-1- | (g/kg) | | | | 438 | 280 |
| propanol (2,3-D) | | | | | | |
| 3-chloro-1,2- | (g/kg) | 0.1 | 0.1 | 0.3 | 0.1 | |
| propanediol | | | | | | |
| Glycerol | (g/kg) | — | 0.1 | 0.1 | — | |
| Chloroethers | (g/kg) | 0.94 | 0.91 | 0.81 | 8.94 | 5.3 |
| H₂O | (g/kg) | 14 | 14 | 20 | 29 | 23 |
| NaCl | (g/kg) | 0.018 | 0.02 | 0.061 | 0.28 | 0.076 |
| Second fraction | | | | | | |
| separated | | | | | | |
| Flow rate | (g/h) | 270.4 | 195.5 | 187.6 | 179.4 | 181 |
| Density | (g/ml) | 1.202 | 1.253 | 1.187 | 1.179 | 1.172 |
| Total Organic | (gC/l) | 8 | 8 | 8.1 | 10 | 12 |
| Carbon | | | | | | |
| Epichlorohydrin | (g/kg) | 12.4 | 12.2 | 11.3 | 11.6 | 9.7 |
| 1,3-dichloro-2- | (g/kg) | 2.09 | 2.1 | 4.4 | 0.02 | 0.06 |
| propanol (1,3-D) | | | | | | |
| 2,3-dichloro-1- | (g/kg) | | | | 0.1 | 4.6 |
| propanol (2,3-D) | | | | | | |
| 3-chloro-1,2- | (g/kg) | 0.16 | 0.14 | 0.15 | 0.1 | 0.1 |
| propanediol | | | | | | |
| Glycerol | (g/kg) | 0 | 0.1 | 0.1 | 0.1 | 0.1 |
| Chloroethers | (g/kg) | | 0.1 | 0.13 | 0.35 | |
| NaCl | (g/kg) | 237 | 239 | 243 | 204 | 245 |
| NaOH | (g/kg) | 0.3 | 1.2 | 0.4 | 20.6 | 0.2 |
| Degree of | (% | 99.8 | 99.2 | 99.7 | 87 | 99.9 |
| conversion of | mol/mol) | | | | | |
| NaOH | | | | | | |
| Degree of overall | (% | 92 | 92 | 83 | 65 | 80 |
| conversion of 1,3-D + | mol/mol) | | | | | |
| 2,3-D | | | | | | |
| EPI selectivity with | (% | 94 | 93 | 96 | 84 | 83 |
| respect to the | mol/mol) | | | | | |
| NaOH consumed | | | | | | |

1- A pocess for manufacturing epichlorohydrin comprising the following steps:
a) in a liquid reaction medium, a mixture of 1,3-dichloro-2-propanol and 2,3-dichloro-1-propanol, in which the 1,3-dichloro-2-propanol content is at least 10 wt %, is reacted with at least one basic compound in order to form epichlorohydrin and a salt; and
b) at least one part of the liquid reaction medium from step a) is subjected to a settling operation in which at least a first fraction containing most of the epichlorohydrin which was contained in the part of the reaction medium from step a) before the settling operation and a second fraction containing most of the salt which was contained in the part of the reaction medium from step a) before the settling operation are separated.

2- The process according to claim 1, wherein the part of the reaction medium from step a) is subjected to a treatment prior to the settling operation from step b), and wherein the treatment is selected from the roup consisting of heating, cooling, dilution, addition of a salt, addition of an acid compound, and combinations of at least two of them.

3- The process according to claim 1, wherein at least one part of the dichloropropanol from step a) is obtained by reaction between glycerol and a chlorinating agent containing hydrogen chloride and/or by reaction between allyl chloride and a hypochlorinating agent and/or by reaction between allyl alcohol and a chlorinating agent and/or by reaction between 2,3-dichloropropionaldehyde and a hydrogenating agent and/or by reaction between 1,2-dichloroethylene and a hydroformylating agent and/or by reaction between 1,3-dichloroacetone and a hydrogenating agent.

4- The process according to claim 1, wherein the basic compound from step a) is selected from the group consisting of alkali and alkaline-earth metal oxides, hydroxides, carbonates, hydrogencarbonates, phosphates, hydrogenphosphates, borates, their aqueous suspensions or solutions, and mixtures thereof, and wherein the salt is selected from the group consisting of alkali and alkaline-earth metal chlorides, sulfates, hydrogensulfates, phosphates, hydrogenphosphates, borates, and mixtures thereof.

5- The process according to claim 1, in which wherein the first fraction separated in step b) comprises at least 100 g of epichlorohydrin/kg of first fraction, and wherein the second fraction separated in step b) comprises at least 50 g of salt/kg of second fraction.

6- The process according to claim 1, wherein the steps a) and b) are carried out in the absence of organic solvent, and wherein the difference in density between the first and the second fraction separated in step b) is at least 0.001.

7- The process according to claim 1, wherein a portion of salt from the second fraction separated in step b) is not produced during the reaction of step a) but is added to step a), and wherein a filtration step is carried out between step a) and step b).

8- The process according to claim 1, wherein the first fraction separated in step b) is used as a reactant in a process for manufacturing epoxy derivatives, glycidyl ethers, glycidyl esters, synthetic glycerol, polyamide-epichlorohydrin resins, products used in food and drink applications including polyacrylamides, polyamines or quaternary ammonium salts, resins for the production of water-resistant paper, epichlorohydrin elastomers including epichlorohydrin homopolymers, epichlorohydrin/ethylene oxide copolymers or epichlorohydrin/ethylene oxide/allyl glycidyl ether terpolymers, surfactants, flame retardants, cationization agents, or detergent ingredients, and/or
wherein the second fraction separated in step b) is used as a reactant in an electrolysis process.

9- The process according to claim 1, wherein steps a) and b) are carried out in continuous mode.

10- The process according to claim 1, wherein the reaction from step a) is carried out at a temperature of at least 0° C. and at most 100° C., at a pressure of at least 0.01 bar absolute and at most 20 bar absolute and over a time of at least 1 min and at most 240 min when step a) is carried out in batch mode or for a residence time of at least 1 min and at most 240 min when step a) is carried out in continuous mode, and wherein the settling of step b) is carried out at a temperature of at least 0° C. and at most 100° C., at a pressure of at least 0.01 bar absolute and at most 20 bar absolute and over a time of at least 5 min and at most 120 min when step a) is carried out in batch mode or for a residence time of at least 5 min and at most 120 min when step a) is carried out in continuous mode.

11- The process according to claim 1, wherein water is added to step a) and/or between step a) and step b) and/or to step b).

12- An aqueous composition of which the salt content is greater than or equal to 50 g/kg of aqueous composition and of which the epichlorohydrin content is at least 0.1 g/kg and at most 60 g/kg of aqueous composition, optionally being obtained according to the process of claim 1, in which case the second fraction separated constitutes the aqueous composition.

13- The aqueous composition according to claim 12,
a) in which the water content is at least 500 g/kg and at most 990 g/kg of aqueous composition;
b) in which the salt is an inorganic salt selected from the group consisting of alkali and alkaline-earth metal chlorides, sulfates, hydrogensulfates, phosphates, hydrogenphosphates, borates, and mixtures thereof;
c) which comprises, in addition, at least one of the compounds selected from the group consisting of:
i. 1,3-dichloro-2-propanol, 2,3-dichloro-1-propanol, and mixtures thereof, and of which the sum of the contents is at least 0.1 g/kg and at most 100 g/kg of aqueous composition;
ii. 3-chloro-1,2-propanediol, 2-chloro-1,3-propanediol, and mixtures thereof, and of which the sum of the contents is at least 0.1 g/kg and at most 50 g/kg of aqueous composition;
iii. glycerol, chloroacetone, hydroxyacetone, glycidol, 2-chloro-2-propen-1-ol and mixtures of at least two of them; and
iv. a basic inorganic compound selected from the group consisting of alkali and alkaline-earth metal oxides, hydroxides, carbonates, hydrogencarbonates, phosphates, hydrogenphosphates, borates, and mixtures of at least two of them, in a content of at least 0.1 g/kg of aqueous composition and at most 25 g/kg;
d) of which the density is at least 1.03 and at most 1.28; and
e) of which the total organic carbon content is at most 40 g C/kg of aqueous composition.

14- An organic composition of which the epichlorohydrin content is at least 100 g/kg and at most 900 g/kg of organic composition and of which the chloroacetone content is at least 0.005 g/kg and at most 2 g/kg of organic composition, optionally being obtained according to the process of claim 1, in which case the first fraction separated constitutes the organic composition.

15- The organic composition according to claim 14, comprising, in addition, at least one of the compounds selected from the group consisting of:
a) acrolein in a content of at least 0.07 g/kg and at most 5 g/kg of organic composition;
b) methyl glycidyl ether in a content of at least 0.005 g/kg and at most 5 g/kg of organic composition;
c) chloroethers of crude chemical formula: C₆H₁₀Cl₂O₂, C₆H₁₂Cl₂O, C₆H₉Cl₁₃O₂, C₆H₁₁Cl₃O₂, or mixtures of at least two of them, and of which the sum of the contents is at least 0.5 g/kg and at most 20 g/kg of organic composition;
d) 1,3-dichloro-2-propanol, 2,3-dichloro-1-propanol, or mixtures thereof, and of which the sum of the contents is at least 90 g/kg and at most 900 g/kg of organic composition;
e) 3-chloro-1,2-propanediol, 2-chloro-1,3-propanediol, or mixtures thereof, and of which the sum of the contents is at least 0.5 g/kg and at most 5 g/kg of organic composition;
f) glycerol, hydroxyacetone, glycidol, or mixtures of at least two of them, and of which the sum of the contents is at least 0.1 g/kg and at most 100 g/kg of organic composition;
g) 1,2,3-trichloropropane, in a content of at least 0.01 g/kg and at most 10 g/kg of organic composition;
h) cis and trans 1,3-dichloropropenes, or mixtures thereof, and of which the sum of the contents is at least 0.01 g/kg and at most 2 g/kg of organic composition;
i) 1,3-dichloropropane, in a content of at least 0.01 g/kg and at most 2 g/kg of organic composition;
j) 2-chloro-2-propen-1-ol, in a content of at the very least 0.01 g/kg and at most 2 g/kg of organic composition;
k) water, in a content of at least 1 g/kg and at most 90 g/kg of organic composition;
l) a salt selected from the group consisting of alkali and alkaline-earth metal chlorides, sulfates, hydrogensulfates, phosphates, hydrogenphosphates, borates, and mixtures of at least two of them, and of which the sum of the contents is at least 0.01 g/kg and at most 10 g/kg of organic composition; and
m) a basic inorganic compound selected from the group consisting of alkali and alkaline-earth metal oxides, hydroxides, carbonates, hydrogencarbonates, phosphates, hydrogenphosphates, borates, and mixtures of at least two of them.

16- A method for carrving out an electrolysis process comprising using the aqueous composition according to claim 12.

17- A process for manufacturing a product selected from the group consisting of epoxy derivatives glycidyl ethers, glycidyl esters, synthetic glycerol, polyamide-epichlorohydrin resins, products used in food and drink applications including polyacrylamides, polyamines or quaternary ammonium salts, resins for the production of water-resistant paper, epichlorohydrin elastomers including epichlorohydrin homopolymers, epichlorohydrin/ethylene oxide copolymers or epichlorohydrin/ethylene oxide/allyl glycidyl ether terpolymers, surfactants, flame retardants, cationization agents, and detergent ingredients, said process comprising using the organic composition according to claim 14.

18- An aqueous composition of which the salt content is greater than or equal to 50 g/kg and of which the epichlorohydrin content is at least 0.1 g/kg and at most 60 g/kg.

19- An organic composition of which the epichlorohydrin content is at least 100 g/kg and at most 900 g/kg of organic composition and of which the chloroacetone content is at least 0.005 g/kg and at most 2 g/kg of organic composition.
